(19) United States
(12) Patent Application Publication
(10) Pub. No.: US 2020/0316113 A1
(43) Pub. Date: Oct. 8, 2020

(54) USE OF COMPOSITION COMPRISING FERROUS AMINO ACID CHELATE IN MANUFACTURE OF MEDICAMENT FOR INHIBITING ANGIOGENESIS

(71) Applicant: PROFEAT BIOTECHNOLOGY CO., LTD., TAOYUAN CITY (TW)

(72) Inventors: Tsun-Yuan LIN, Taoyuan City (TW); Mu-Kuei CHEN, Taoyuan City (TW); Hsun-Jin JAN, Taoyuan City (TW); Chai-Hui FU, Taoyuan City (TW)

(21) Appl. No.: 16/957,215

(22) PCT Filed: Dec. 29, 2017
(86) PCT No.: PCT/CN2017/119620

§ 371 Date: Jun. 23, 2020

(51) Int. Cl. A61K 33/26; A61K 31/197; A61K 47/12

(52) U.S. Cl. CPC A61K 33/26; A61K 9/0053; A61K 47/12; A61K 31/197

(57) ABSTRACT

The present invention is related to a use of a composition comprising ferrous amino acid chelate for the manufacture of a medicament for inhibiting angiogenesis, wherein the medicament comprises an effective amount of the ferrous amino acid chelate composition and a pharmaceutically acceptable carrier. The amino acid can be glycine and the angiogenesis can be related to cancer or eye disease.

### CROSS REFERENCE

This application is a 35 U.S.C. § 371 national phase application of International Application Serial No. PCT/CN2017/119620, filed Dec. 29, 2017, the entire content of which is incorporated by reference herein.

### BACKGROUND OF THE INVENTION

### 1. Field of the Invention

The present invention relates to a use of a composition comprising ferrous amino acid chelate, and particularly the use for the manufacture of a medicament for inhibiting angiogenesis.

### 2. Description of the Prior Arts

In the human body, angiogenesis is a process involving the slow migration, growth and differentiation of the cells in the inner wall of blood vessels. Angiogenesis can be induced by a variety of chemicals released from extravascular cells, such as vascular endothelial growth factor (VEGF).

Angiogenesis is an important mechanism in the human body. When more blood vessels are needed due to the hypoxia of the tissues, the secretion of VEGF is increased to raise the possibility of new blood vessel growth. The angiogenesis can be caused by cancer metastasis, diabetic retinopathy, high myopic retinopathy, or age-related retinopathy. Conventionally, Avastin® (bevacizumab) is a recombinant humanized monoclonal antibody, which selectively binds to VEGF and to the receptors (Flt-1 and KDR) located on the surface of endothelial cells. The neutralization of the bioactivity of VEGF reduces tumor vessel formation, so the tumor growth is inhibited.

In protein therapies, however, the protein cannot be delivered to the site in need effectively by oral administration, intravenous injection, artery injection, or muscle injection due to the molecular weight and electric charge of the protein. Therefore, the protein is likely to be eliminated or metabolized during delivery, or be delivered to the tissues not in need and this results in a waste.

In view of this, it is necessary to improve the existing technology and develop a medicament for inhibiting angiogenesis which can be easily delivered.

### SUMMARY OF THE INVENTION

To overcome the shortcomings, the present invention provides a use of a composition comprising ferrous amino acid chelate in the manufacture of a medicament for inhibiting angiogenesis, wherein the composition comprising the ferrous amino acid chelate has the effect of inhibiting angiogenesis.

To achieve the above purpose, the present invention provides a use of a composition comprising ferrous amino acid chelate in the manufacture of medicament for inhibiting angiogenesis, wherein the medicament comprises the effective amount of the ferrous amino acid chelate composition and a pharmaceutically acceptable carrier.

According to the present invention, the “composition comprising ferrous amino acid chelate” refers to a composition comprising ferrous amino acid chelate made by mixing inorganic iron and amino acid.

Preferably, the chelating ratio of ferrous to amino acid of the ferrous amino acid chelate in the composition comprising the ferrous amino acid chelate is between 1:1 and 1:4.

Preferably, the chelating ratio of ferrous to amino acid of the ferrous amino acid chelate in the composition comprising the ferrous amino acid chelate is between 1:1.5 and 1:2.5.

Preferably, the effective amount of the composition comprising the ferrous amino acid chelate for mice is between 0.2 milligrams per kilogram per day (mg/kg/day) and 15 mg/kg/day. Preferably, it is between 0.3 mg/kg/day and 14 mg/kg/day. More preferably, it is between 0.4 mg/kg/day and 12 mg/kg/day. Preferably, the effective amount of the composition comprising the ferrous amino acid chelate for humans is between 0.016 mg/kg/day and 1.22 mg/kg/day. Preferably, it is between 0.024 mg/kg/day and 1.14 mg/kg/day. More preferably, it is between 0.032 mg/kg/day and 0.98 mg/kg/day. The above dosages are calculated in accordance with the guidance document “Estimating the Maximum Safe Starting Dose in Initial Clinical Trials for Therapeutics in Adult Healthy Volunteers” published by the U.S. Food and Drug Administration in 2005.

Preferably, the composition comprising the ferrous amino acid chelate is prepared by mixing inorganic iron and amino acid and heating at 60° C. to 90° C. for 8 hours to 48 hours to obtain the composition comprising the ferrous amino acid chelate, wherein the weight ratio of inorganic iron to amino acid is between 1:1.2 and 1:1.5.

More preferably, the inorganic iron is ferrous sulfate, ferrous chloride, ferrous pyrophosphate, or any combination thereof. More preferably, the amino acid is glycine.

More preferably, the composition comprising the ferrous amino acid chelate contains 95 wt % to 100 wt % of ferrous glycinate chelate. More preferably, the composition comprising the ferrous amino acid chelate contains 98 wt % to 99.9 wt % of ferrous glycinate chelate.

According to the present invention, the “effective amount” refers to a dosage which effectively achieves desired angiogenesis inhibition during a required period of time. According to the present invention, it refers to a specific range of amounts of the composition comprising the ferrous amino acid chelate which inhibits the migration, reduces the invasion, or inhibits the tube formation of human umbilical vein endothelial cells (HUVECs) after administration. According to the present invention, it also refers to a dosage which effectively inhibits the angiogenesis.

According to the present invention, the “pharmaceutically acceptable carrier” includes, but is not limited to, reducing agents, solvents, emulsifiers, suspending agents, decomposers, binding agents, excipients, stabilizing agents, chelating agents, diluents, gelling agents, preservatives, lubricants, surfactants and other similar carriers or the carriers that are suitable for the present invention.

Preferably, the reducing agents include, but are not limited to, ascorbic acid, citric acid, acetic acid, propionic acid, butyric acid, lactic acid, malic acid, sulfonic acid, succinic acid, or any combination thereof.

In accordance with the present invention, the “medicament” can be prepared in various forms, including, but not limited to, liquid, semi-solid and solid dosage forms, such as solutions, emulsions, suspensions, powders, tablets, pills, lozenges, troches, chewing gums, capsules, liposomes, suppositories, and other similar dosage forms or the dosage forms that are suitable for the present invention.

Preferably, the medicament is in an enteral or parenteral dosage form.

More preferably, said enteral dosage form is an oral dosage form, including, but not limited to, solutions, emulsions, suspensions, powders, tablets, pills, lozenges, troches, chewing gums, or capsules.

Preferably, said angiogenesis is related to, including, but not limited to, cancer or eye disease.

More preferably, said cancer includes, but is not limited to, melanoma, liver cancer, colon cancer, lung cancer, gastric cancer, esophageal cancer, brain tumor, head and neck cancer, esophageal cancer, chest wall tumor, thymoma, mediastinal tumor, breast cancer, abdomen-pelvis tumor, gallbladder cancer, biliary tract cancer, pancreatic cancer, small intestinal tumor, large intestinal tumor, anal cancer, bladder cancer, renal cell carcinoma, cervix cancer, endometrial cancer, ovarian cancer, uterine sarcoma, prostate cancer, leukemia, or skin cancer.

More preferably, said liver cancer includes, but is not limited to, hepatoma or liver adenocarcinoma.

More preferably, said lung cancer includes, but is not limited to, small cell lung cancer or non-small cell lung cancer (NSCLC).

More preferably, said brain tumor includes, but is not limited to, low-grade astrocytoma, high-grade astrocytoma, pituitary adenoma, meningioma, CNS lymphoma, oligodendroglioma, craniopharyngioma, ependymoma, or glioma.

More preferably, said head and neck cancer includes, but is not limited to, laryngeal cancer, oropharyngeal cancer, nasopharyngeal tumor, salivary gland tumor, hypopharyngeal cancer, thyroid cancer, or oral cavity tumor.

More preferably, said eye disease includes, but is not limited to, diabetic retinopathy, diabetic macular edema, age-related macular degeneration, juvenile macular degeneration, corneal neovascularization, choroidal neovascularization (CNV), retinopathy of prematurity (ROP), retinitis pigmentosa (RP), trachoma, glaucoma, xerophthalmia, neurological eye disease, retinal artery occlusion, uveitis, choroiditis, central serous chorioretinopathy, central exudative chorioretinopathy, polypoidal choroidal vasculopathy, or complication of laser eye surgery.

The present invention provides a use of a composition comprising ferrous amino acid chelate in the manufacture of medicament for treating angiogenesis-related diseases, wherein the medicament comprises an effective amount of ferrous amino acid chelate composition and a pharmaceutically acceptable carrier.

Preferably, said angiogenesis-related disease includes, but is not limited to, cancer or eye disease.

One advantage of the present invention is that the composition comprising ferrous amino acid chelate of the present invention can effectively prevent cell migration, cell invasion, and tube formation of HUVEC induced by cancer cells, so as to effectively inhibit the angiogenesis. Besides, Composition A1 also can effectively prevent the migration, invasion, and tube formation of HUVEC induced by VEGF, so as to effectively inhibit the angiogenesis.

### BRIEF DESCRIPTION OF THE DRAWINGS

FIG. 1 is a line graph showing the values of OD 565 of human umbilical vein endothelial cells (HUVECs) with administration of 50 μg/mL and 100 μg/mL Composition A1 of the present invention and control group detected by MTT assay.

FIG. 2 is a bar chart showing the values of OD 565 of HUVECs with administration of 50 μg/mL and 100 μg/mL Composition A1 of the present invention and control group detected by MTT assay (with the value of control group at each time point set as the basis).

FIG. 3 is photos showing tube formation of HUVECs with administration of 50 μg/mL and 100 μg/mL Composition A1 of the present invention and control group.

FIG. 4 is cell staining photos showing cell migration of HUVECs in control group or positive control group or administered with 10 μg/mL, 25 μg/mL, 50 μg/mL, and 100 μg/mL Composition A1 of the present invention with the induction of conditioned medium.

FIG. 5 is a bar chart showing cell migration of HUVECs in control group or administered with 10 μg/mL, 25 μg/mL, 50 μg/mL, and 100 μg/mL Composition A1 of the present invention with the induction of conditioned medium (with the cell number of control group set as the basis).

FIG. 6 is cell staining photos showing cell invasion of HUVECs in control group or administered with 10 μg/mL, 25 μg/mL, 50 μg/mL, and 100 μg/mL Composition A1 of the present invention with the induction of conditioned medium.

FIG. 7 is a bar chart showing cell invasion of HUVECs in control group or administered with 25 μg/mL, 50 μg/mL, and 100 μg/mL Composition A1 of the present invention with the induction of conditioned medium (with the cell number of control group set as the basis).

FIG. 8 is photos showing tube formation of HUVECs in control group or administrated with 50 μg/mL and 100 μg/mL Composition A1 of the present invention with the induction of conditioned medium.

FIG. 9 is a line graph showing the values of OD 565 of HUVECs in control group or administered with 1 ng/mL, 5 ng/mL, 10 ng/mL, 20 ng/mL VEGF detected by MTT assay.

FIG. 10 is a bar chart showing the cell migration of HUVECs in control group or administered with 50 μg/mL, 100 μg/mL, 250 μg/mL, and 500 μg/mL Composition A1 of the present invention with the induction of VEGF (with the cell number of control group set as the basis).

### DETAILED DESCRIPTION OF THE PREFERRED EMBODIMENTS

The technical features adopted in the present invention in order to achieve the purpose are further explained through the preferred embodiments below and the accompanying figures.

### Preparation Example 1: Preparation of the Composition Comprising Ferrous Amino Acid Chelate

The composition comprising ferrous amino acid chelate was prepared as follows. Ferrous sulfate and glycine (with a purity of more than 98%) were mixed at a weight ratio of 1:1.3 and heated at 60° C. to 90° C. for 8 hours to 48 hours to obtain the composition comprising ferrous amino acid chelate, wherein the chelating ratio of ferrous to amino acid of the ferrous amino acid chelate was between 1:1 and 1:4. Said composition was referred to as Composition A1.

### Preparation Example 2: Collection of the Conditioned Medium

The conditioned medium of MDA-MB-231 breast cancer cells was collected: (1) 3×10⁵ cells were seeded in a 6-well culture plate and left overnight; (2) the cells were washed with phosphate buffered saline (PBS) once, and then were cultured with a serum-free Roswell park memorial institute-1641 (RPMI 1640) medium in an incubator under 37° C. for 48 hours; (3) the medium after cell culture was collected and then subjected to low speed centrifugation for 5 min, and the supernatant was collected as conditioned medium.

### Example 1: The Effect of Composition A1 on Cell Proliferation

The HUVECs were used in the experiments. The cells were seeded in a 24-well plate at a density of 2×10⁴ cells/well. The experiments include control group (not treated with Composition A1) and the groups were respectively treated with 50 μg/mL Composition A1 (from Preparation Example 1), and 100 μg/mL Composition A1 (from Preparation Example 1) for 0 hour, 24 hours, 48 hours and 72 hours. Each group was triplicated. The MTT assays were performed and values of OD 565 were measured in order to observe the effect of Composition A1 on the cell growth.

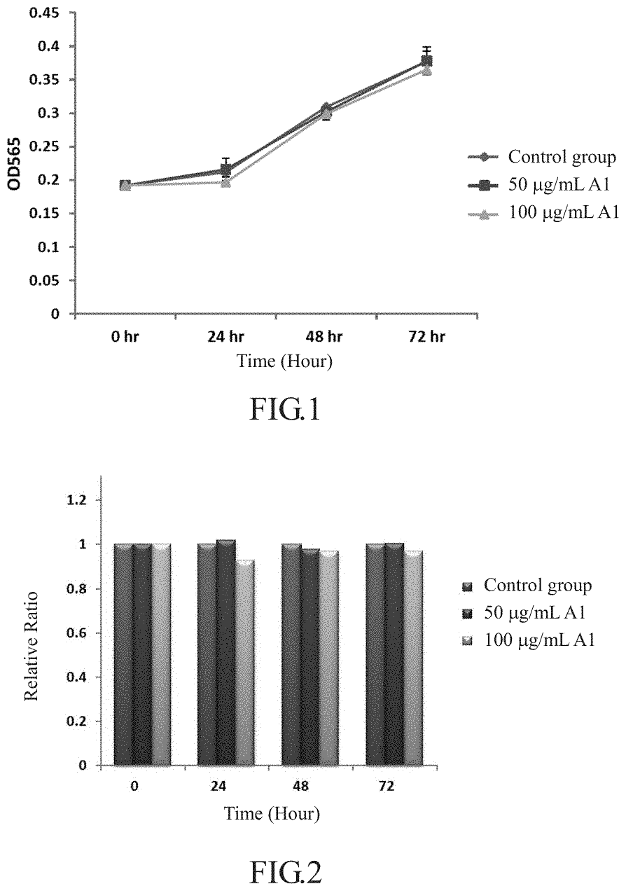

As shown in FIG. 1 and FIG. 2, with the control group used as the basis, neither 50 μg/mL Composition A1 nor 100 μg/mL Composition A1 treatment for 0 hour, 24 hours, 48 hours and 72 hours showed a significant effect on cell growth of HUVEC.

### Example 2: The Effect of Composition A1 on Tube Formation

Tube formation assay: (1) the matrigel was thawed under 4° C. overnight; (2) HUVECs were collected and washed with PBS once, suspended with M199 medium containing 0.5% FBS, and then starved in an incubator under 37° C. for 2 hours; (3) a 96-well plate was placed on ice, added with 60 μL of completely thawed matrigel, and then was placed in an incubator under 37° C. for more than 1 hour for gelling; (4) the HUVECs after starvation were collected with a concentration of 2×10⁵ cells/mL, and then subjected to low-speed centrifugation for 5 min to remove the medium; (5) the cells after centrifugation were homogeneously suspended in 500 μL medium containing different dosages of A1 as follows:

a) control group: serum-free M199 medium;

b) 50 μg/mL A1 group: serum-free M199 medium containing 50 μg/mL Composition A1;

c) 100 μg/mL A1 group: serum-free M199 medium containing 100 μg/mL Composition A1;

100 μL cell suspension from each above-mentioned group was respectively added to the matrigel-coated 96-well plate (in which every group was triplicated); and (6) the 96-well plate was placed in an incubator under 37° C. and observed for 4 hours.

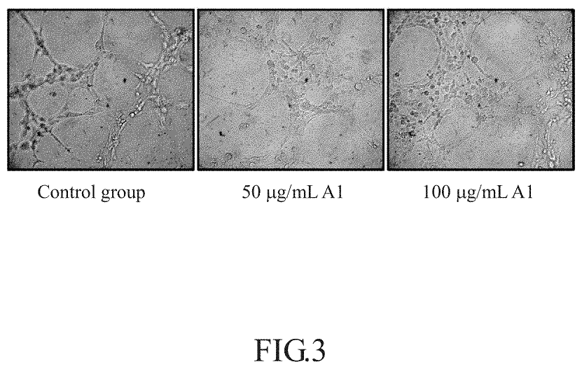

As shown in FIG. 3, ring-like structures formed by a monolayer of cells which were the characteristics of the angiogenesis were observed in control group. In contrast, the cells were aggregative and the ring-like structures formed by a monolayer of cells did not appear in both 50 μg/mL Composition A1 group and 100 μg/mL Composition A1 group. Therefore, Composition A1 can be used for inhibiting the tube formation of HUVEC.

### Example 3: The Effect of Composition A1 on Cell Migration Induced by Conditioned Medium

Cell migration assay: (1) HUVECs were collected, washed with PBS once, suspended with 1 mL M199 medium containing 1% fetal bovine serum (FBS), and then starved in an incubator under 37° C. for 2 hours; (2) 300 μL of the cell suspension medium containing 1×10⁵ HUVECs were seeded into upper chambers, and added with 1 mL medium containing different dosages of A1 as follows:

a) positive control group: M199 containing 1% FBS; and then the upper chamber was placed into a 24-well plate, in which 600 μL M199 containing 10% FBS is added to corresponding wells;

b) control group: M199 medium containing 1% FBS; and then the upper chamber was placed into the 24-well plate, in which 600 μL conditioned medium (from Preparation Example 2) is added to corresponding wells;

c) 10 μg/mL A1 group: M199 medium containing 10 μg/mL Composition A1 and 1% FBS; and then the upper chamber was placed into the 24-well plate, in which 600 μL conditioned medium (from Preparation Example 2) is added to corresponding wells;

d) 25 μg/mL A1 group: M199 medium containing 25 μg/mL Composition A1 and 1% FBS; and then the upper chamber was placed into the 24-well plate, in which 600 μL conditioned medium (from Preparation Example 2) is added to corresponding wells;

e) 50 μg/mL A1 group: M199 medium containing 50 μg/mL Composition A1 and 1% FBS; and then the upper chamber was placed into the 24-well plate, in which 600 μL conditioned medium is added to corresponding wells (of Preparation Example 2);

f) 100 μg/mL A1 group: M199 medium containing 100 μg/mL Composition A1 and 1% FBS; and then the upper chambers were placed into the 24-well plate, in which 600 μL conditioned medium (of Preparation Example 2) is added to corresponding wells;

(3) the 24-well plate with upper chambers was placed in an incubator under 37° C. for 4 hours for cell migration; (4) the upper chambers were taken out, and were immersed in methanol for 8 minutes for cell fixation after removing the medium, and then were taken out and air-dried; (5) the cells in the upper chambers were stained with 10× diluted Giemsa solution, and the upper surfaces of the bottoms of the upper chambers were wiped clean by cotton swabs; and (6) the migrating cells were counted.

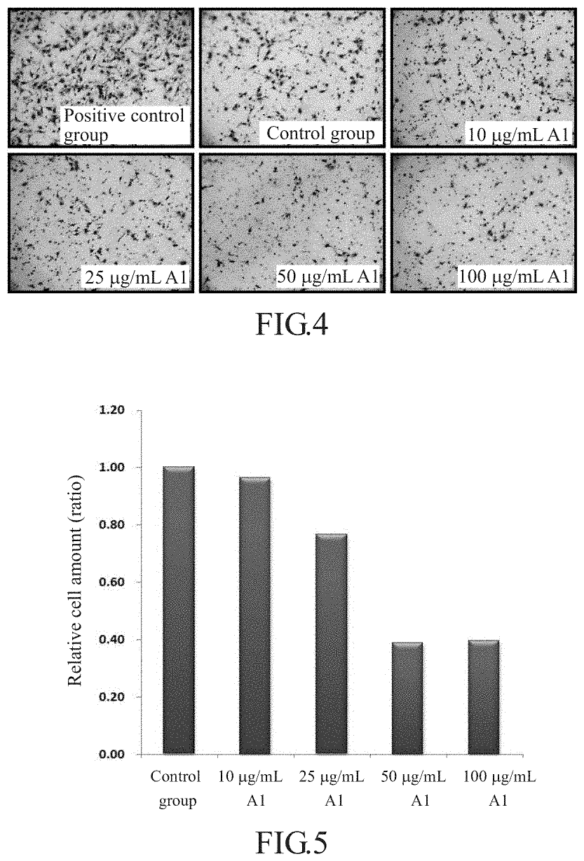

As shown in FIG. 4 and FIG. 5, with the control group used as the basis, the higher the concentration of Composition A1 is, the lower the number of the migrating cells is. Especially, the numbers of the migrating cells in the 50 μg/mL A1 group and 100 μg/mL A1 group were about 60% lower than that of the control group. Therefore, Composition A1 can inhibit the migration of HUVEC, and can inhibit the cell migration under the influence of the conditioned medium.

### Example 4: The Effect of Composition A1 on Cell Invasion Induced by Conditioned Medium

Cell invasion assay: (1) the invasion chambers were placed under room temperature; (2) HUVECs were collected, washed with PBS once, suspended with 1 mL M199 medium containing 1% FBS, and then starved in an incubator under 37° C. for 2 hours; (3) the invasion chambers were added with 500 μL serum-free medium, and were placed in an incubator under 37° C. for 2 hours to rehydrate the matrigel in the invasion chambers; (4) 300 μL of the cell suspension containing 5×10⁴ HUVECs were seeded into the invasion chambers, and added with 1 mL medium containing different dosages of A1 as follows:

a) control group: M199 medium containing 1% FBS;

b) 10 μg/mL A1 group: M199 medium containing 10 μg/mL Composition A1 and 1% FBS;

c) 25 μg/mL A1 group: M199 medium containing 25 μg/mL Composition A1 and 1% FBS;

d) 50 μg/mL A1 group: M199 medium containing 50 μg/mL Composition A1 and 1% FBS;

e) 100 μg/mL A1 group: M199 medium containing 100 μg/mL Composition A1 and 1% FBS;

and then the invasion chambers were placed into a 24-well plate with 600 μL conditioned medium (from Preparation Example 2); (5) the 24-well plate with invasion chambers was placed in an incubator under 37° C. for 16 hours for cellinvasion; (6) the invasion chambers were taken out, and were immersed in methanol for 8 minutes for cell fixation after removing the medium, and then were taken out and air-dried; (7) the cells in the invasion chambers were stained with 10× diluted Giemsa solution for 1 hour, and the upper surfaces of the bottoms of the invasion chambers were wiped clean by cotton swabs; and (8) the invading cells were counted.

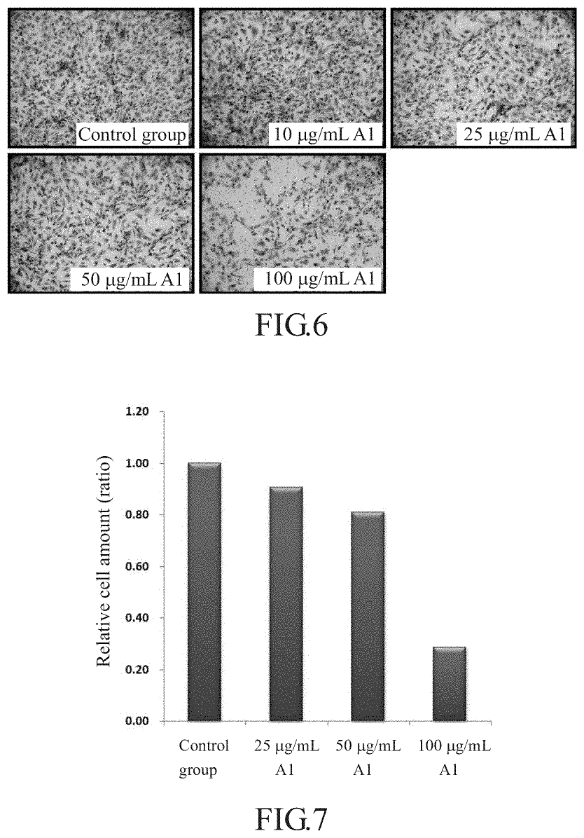

As shown in FIG. 6 and FIG. 7, with the control group used as the basis, the higher the concentration of Composition A1 is, the lower the number of the invading cells is. Especially, the number of the invading cells in the 100 μg/mL A1 group was about 70% lower than that in the control group. Therefore, Composition A1 can inhibit the invasion of HUVECs and can inhibit the cell invasion under the influence of the conditioned medium.

### Example 5: The Effect of Composition A1 on Tube Formation Induced by Conditioned Medium

Tube formation assay: (1) the matrigel was thawed under 4° C. overnight; (2) HUVECs were collected and washed with PBS once, suspended with M199 medium containing 0.5% FBS, and then starved in an incubator under 37° C. for 2 hours; (3) a 96-well plate was placed on ice, added with 60 μL of completely thawed matrigel, and then was placed in an incubator under 37° C. for more than 1 hour for gelling; (4) the HUVECs after starvation were collected with a concentration of 2×10⁴ cells/mL, and then subjected to low-speed centrifugation for 5 min to remove the medium; (5) the cells after centrifugation were homogeneously suspended in 500 μL medium as follows:

a) control group: conditioned medium;

b) 50 μg/mL A1 group: conditioned medium containing 50 μg/mL Composition A1;

c) 100 μg/mL A1 group: conditioned medium containing 100 μg/mL Composition A1;

100 μL cell suspension from each above-mentioned group was respectively added to the matrigel-coated 96-well plate. (in which every group was triplicated); and (6) the 96-well plate was placed in an incubator under 37° C. and observed for 3 hours.

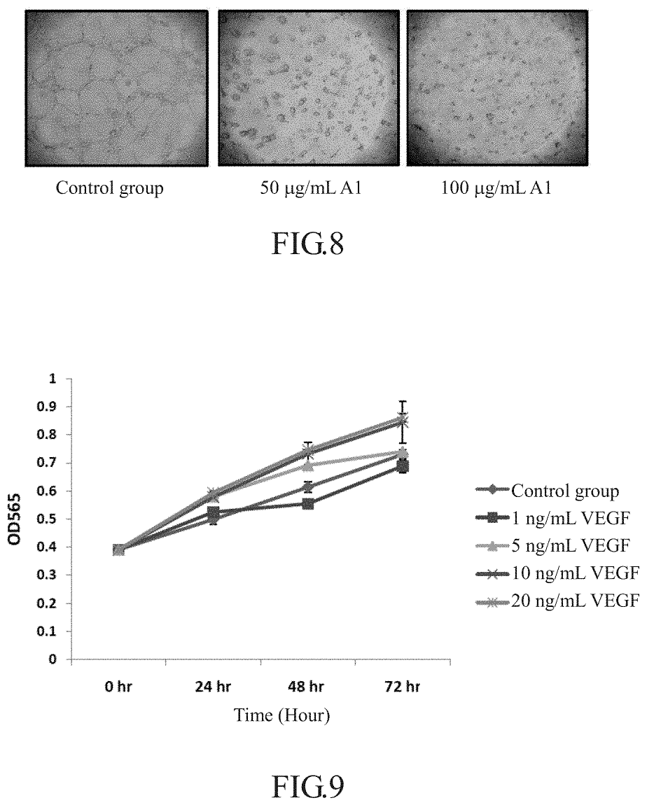

As shown in FIG. 8, ring-like structures formed by a monolayer of cells which were the characteristics of the angiogenesis were observed in control group. In contrast, the cells dispersed in both 50 μg/mL Composition A1 group and 100 μg/mL Composition A1 group. Therefore, Composition A1 can be used for inhibiting the tube formation of HUVEC induced by cancer cells.

To sum up, Composition A1 has the effect of preventing cell migration, cell invasion and tube formation of HUVECs induced by cancer cells, so as to have the effect of inhibiting the angiogenesis.

### Example 6: The Effect of VEGF and Composition A1 on Cell Proliferation

Recombinant human vascular endothelial growth factor (VEGF) was purchased from R&D systems; Cat No. 293-VE. The HUVECs were used in the experiments. The cells were seeded in a 24-well plate at a density of 2×10⁴ cells/well. The experiments include control group (not treated with VEGF) and the groups respectively treated with 1 ng/mL VEGF, 5 ng/mL VEGF, 10 ng/mL VEGF and 20 ng/mL VEGF for 0 hour, 24 hours, 48 hours and 72 hours. Each group was triplicated. The MTT assays were performed and values of OD 565 were measured in order to observe the effect of VEGF on cell growth.

As shown in FIG. 9, with control group used as the basis, the cell number of the high concentration groups including 10 ng/mL VEGF group and 20 ng/mL VEGF group was increased because VEGF has the effect of promoting cell proliferation. The influences on the cell proliferation in other concentration groups including 1 ng/mL VEGF group and 5 ng/mL VEGF group were not significant.

Furthermore, when the cells were pre-treated with 0 μg/mL, 50 μg/mL or 100 μg/mL Composition A1 for 24 hours and then treated with 10 ng/mL VEGF for 0 hour, 24 hours, 48 hours, and 72 hours, the cell growth rate of each group at each time point shows no significant influence.

### Example 7: The Effect of Composition A1 on Cell Migration Induced by VEGF

Cell migration assay: (1) HUVECs were collected, washed with PBS once, suspended with 1 mL M199 medium containing 1% FBS, and then starved in an incubator under 37° C. for 2 hours; (2) 300 μL of the cell suspension containing 1×10⁵ HUVECs were collected and seeded into upper chambers, and added with 1 mL medium containing different dosages of A1 as follows:

a) control group: M199 medium containing 1% FBS;

b) 50 μg/mL A1 group: M199 medium containing 50 μg/mL Composition A1 and 1% FBS;

c) 100 μg/mL A1 group: M199 medium containing 100 μg/mL Composition A1 and 1% FBS;

d) 250 μg/mL A1 group: M199 medium containing 250 μg/mL Composition A1 and 1% FBS;

e) 500 μg/mL A1 group: M199 medium containing 500 μg/mL Composition A1 and 1% FBS;

and then the upper chambers were placed into a 24-well plate with serum-free M199 medium containing 10 ng/mL VEGF recombinant protein; (3) the 24-well plate with upper chambers was placed in an incubator under 37° C. for 4 hours for cell migration; (4) the upper chambers were taken out, and were immersed in methanol for 8 minutes for cell fixation after removing the medium, and then were taken out and air-dried; (5) the cells in the upper chambers were stained with 10× diluted Giemsa solution, and the upper surfaces of the bottoms of the upper chambers were wiped clean by cotton swabs; and (6) the migrating cells were counted.

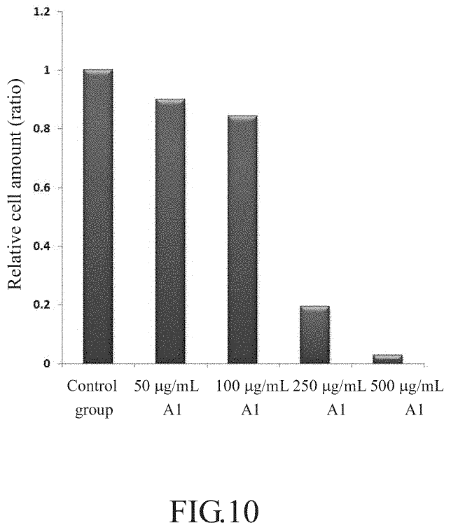

As shown in FIG. 10, with control group used as the basis, the higher the concentration of Composition A1 is, the lower the number of the migrating cells is. Especially, the numbers of the migrating cells in 250 μg/mL A1 group and 500 μg/mL A1 group were about 80% lower than that in the control group. Therefore, Composition A1 can inhibit the migration of HUVECs. In other words, the cell migration of HUVECs induced by VEGF is inhibited.

To sum up, Composition A1 has the effect of preventing cell migration, cell invasion and tube formation of HUVECs induced by VEGF, so as to have the effect of inhibiting the angiogenesis.

It is obvious to a person having ordinary skill in the art that any amendment and modification according to the present invention are not departing from the scope and the spirit of the present invention. Although the preferred embodiments are disclosed in the present invention, it should be understood that the present invention should not be unduly limited to the specific embodiments. In fact, any simple modifications and changes of the above embodiments of the present invention, which are obvious to a person having ordinary skill in the art, are included in the claims.

What is claimed is:

1. A method for inhibiting angiogenesis comprising administering to a subject in need thereof a medicament comprising an effective amount of a composition comprising ferrous amino acid chelate and a pharmaceutically acceptable carrier.

2. The method according to claim 1, wherein the chelating ratio of ferrous to amino acid of the ferrous amino acid chelate in the composition comprising the ferrous amino acid chelate is between 1:1 and 1:4.

3. The method according to claim 1, wherein the chelating ratio of ferrous to amino acid of the ferrous amino acid chelate in the composition comprising the ferrous amino acid chelate is between 1:1.5 and 1:2.5.

4. The method according to claim 1, wherein the effective amount of the composition comprising the ferrous amino acid chelate is between 0.016 mg/kg/day and 1.22 mg/kg/day.

5. The method according to claim 1, wherein the composition comprising the ferrous amino acid chelate is prepared by mixing inorganic iron and amino acid and heating at 60° C. to 90° C. for 8 hours to 48 hours, wherein the weight ratio of inorganic iron to amino acid is between 1:1.2 and 1:1.5.

6. The method according to claim 5, wherein the inorganic iron is ferrous sulfate, ferrous chloride, ferrous pyrophosphate, or any combination thereof; and the amino acid is glycine.

7. The method according to claim 5, wherein the pharmaceutically acceptable carrier comprised in the medicament includes a reductant, wherein the reductant is ascorbic acid, citric acid, acetic acid, propionic acid, butyric acid, lactic acid, malic acid, sulfonic acid, succinic acid, or any combination thereof.

8. The method according to claim 1, wherein the medicament is in an enteral or parenteral dosage form.

9. The method according to claim 8, wherein the enteral dosage form is an oral dosage form, wherein the oral dosage form is a solution, an emulsion, a suspension, powder, a tablet, a pill, a lozenge, a troche, chewing gum, or a capsule.

10. The method according to claim 1, wherein the angiogenesis is related to cancer or eye disease.
